(19) United States
(12) Patent Application Publication
(10) Pub. No.: US 2011/0178011 A1
(43) Pub. Date: Jul. 21, 2011

(54) Polysaccharide/BMP complexes which are soluble at physiological pH

(75) Inventors: Remi SOULA, Lyon (FR); Olivier SOULA, Meyzieu (FR); Gerard SOULA, Meyzieu (FR); Richard CHARVET, Rillieux La Pape (FR); David DURACHER, Lyon (FR)

(73) Assignee: ADOCIA, Lyon (FR)

(21) Appl. No.: 12/950,616

(22) Filed: Nov. 19, 2010

Related U.S. Application Data
(60) Provisional application No. 61/302,823, filed on Feb. 9, 2010.

(30) Foreign Application Priority Data
Nov. 19, 2009 (WO) FR2009/001332
Feb. 9, 2010 (FR) 10/00537

(51) Int. Cl. A61K 38/18; C07K 14/51

(52) U.S. Cl. 514/8.8; 530/397

(57) ABSTRACT

A complex of a polysaccharide and recombinant human BMP-2 and BMP-7, soluble at physiological pH, wherein the polysaccharide/BMP mass ratio is less than 15, the polysaccharide being selected from the group of polysaccharides having carboxyl functional groups, at least one of which is substituted with at least one hydrophobic radical.

### BACKGROUND

The present invention relates to the field of the formulation of bone morphogenetic proteins, BMP-7 and BMP-2.

Bone morphogenetic proteins (BMPs) are growth factors involved in the mechanisms of bone and cartilage formation. BMPs, also known as osteogenic proteins (OPs), were initially characterized by Urist in 1965 (Urist M R. Science 1965; 150, 893). These proteins, isolated from cortical bone, have the ability to induce bone formation in a large number of animals (Urist M R. Science 1965; 150, 893).

BMPs are expressed in the form of propetides which, after post-translational maturation, have a length of between 104 and 139 residues. They possess great sequence homology with respect to one another and have similar three-dimensional structures. In particular, they have 6 cysteine residues involved in intramolecular disulfide bridges forming a “cysteine knot” (Scheufler C. 2004 J. Mol. Biol. 1999; 287, 103; Schlunegger M P, J. Mol. Biol. 1993; 231, 445). Some of them have a 7^{th} cysteine also involved in an intermolecular disulfide bridge responsible for the formation of the dimer (Scheufler C. 2004 J. Mol. Biol. 1999; 287:103).

In their active form, BMPs assemble as homodimers, or even heterodimers, as has been described by Israel et al. (Israel D I, Growth Factors. 1996; 13(3-4), 291). Dimeric BMPs interact with BMPR transmembrane receptors (Mundy et al. Growth Factors, 2004, 22 (4), 233). This recognition is responsible for an intracellular signaling cascade involving, in particular, Smad proteins, thus resulting in the target gene activation or repression.

Some recombinant human BMPs, and in particular rhBMP-2 and rhBMP-7, have clearly shown an ability to induce bone formation in vivo in humans and have been approved for some medical uses.

Thus, recombinant human BMP-2, dibotermin alfa according to the international nonproprietary name, is formulated in products sold under the name InFUSE® in the United States and InductOs® in Europe. This product is prescribed in the fusion of lumbar vertebrae and bone regeneration in the tibia for “nonunion” fractures. In the case of InFUSE® for the fusion of lumbar vertebrae, the surgical procedure consists, first of all, in soaking a collagen sponge with a solution of rhBMP-2, and then in placing the sponge in a hollow cage, LT cage, preimplanted between the vertebrae.

BMP-7, eptotermin alfa according to the international nonproprietary name, plays a direct and indirect role on the differentiation of mesenchymal cells, causing them to differentiate into osteoblasts (Cheng H., J. Bone and Joint Surgery, 2003, 85A, 1544-1552). Thus, recombinant human BMP-7 constitutes the basis of two products: OP-1 Implant for open fractures of the tibia and OP-1 Putty for the fusion of lumbar vertebrae. OP-1 Implant is composed of a powder containing rhBMP-7 and collagen, to be taken up in a 0.9% saline solution. The paste obtained is subsequently applied to the fracture during a surgical procedure. OP-1 Putty is in the form of two powders: one containing rhBMP-7 and collagen, the other containing carboxymethylcellulose (CMC). During a surgical procedure, the CMC is reconstituted with a 0.9% saline solution and mixed with the rhBMP-7 and the collagen. The resulting paste is applied to the site to be treated. Nevertheless, these BMP-7-based products have been the subject of only one limited approval on the part of the FDA, since they have the status of a humanitarian product. The major reasons for this limited approval are an effectiveness that is slightly inferior to an autograft, considered to be the reference treatment (gold standard) and a strong production of antibodies directed against BMP-7.

In addition to the role of BMP-7 in bone growth, it has been demonstrated that BMP-7 plays an important role in cartilage growth and repair. Animal studies demonstrate that OP-1 allows cartilage repair among the various models of lesions of this cartilage, in addition to cartilaginous lesions, arthrosis lesions and intervertebral disk degeneration lesions (Chubinskaya, S. et al., Int. Orthop. 2007, 31 (6), 773-781).

Finally, another established major role of BMP-7 relates to renal growth, since BMP-7 is a morphogen that is essential for the conversion of mesenchymal cells to epithelial cells during kidney development. This property has found a potential therapeutic application in the repair of kidneys damaged by chronic kidney fibrosis (Zeisberg, M. et al., J Biol Chem 2005, 280 (9), 8094-8100), (Sugimoto H. et al. Faseb 2007, 21, 256-264).

Many other applications have been described in the literature, such as the use thereof for liver regeneration (Kinoshita, K. et al. Gut 2007, 56, 706-714; Gessner, O. A. et al. Journal of gastroenterology and hepatology 2008, 23, 1024-1035), corneal regeneration (Saika S. et al. Laboratory Investigation 2005, 85, 474-486), and also for treating strokes (Chang C—F et al., Stroke 2003, 34, 558-564), myocardial infarction (Zeisberg E. M. et al. Nature medecine 2007, 13, N° 8, 952-961), chronic obstructive pulmonary diseases (Myllârniemi M. et al. Am J respir Crit. Care Med 2008, 177, 321-329), spinal cord lesions (De Rivero Vaccari, J. P. et al. Neuroscience letters 2009, 465, 226-229), Parkinson's disease (Harvey B. K. et al. Brain Research 2004, 1022, 88-95), and also critical lower limb ischemia (Moreno-Miralles I. et al. Curr Opi Hematol 2009, 16, 195-201; David L. et al. Cytokines & Growth Factors reviews 2009, 20, 203-212).

### SUMMARY

However, for all these applications, it is necessary to solve the problem of the low solubility of BMP-7 at physiological pH, which results in aggregation of this protein. The low solubility of BMP-7 under physiological conditions and the formation of aggregates makes its use problematic for local applications since the bioavailability of the active protein is reduced. This low solubility of BMP-7 under physiological conditions is even more problematic for systemic applications of BMP-7, whether intravenously or subcutaneously, since the precipitation of BMP-7 at the injection site can result in side effects. Furthermore, it is known that the formation of protein aggregates results in an immunological reaction involving antibody formation.

In the case of BMP-7, the appearance of these immunological reactions is dependent on the site of administration of the formulation. Thus, immunological reactions are observed when BMP-7 is used for posterolateral fusion of lumbar vertebrae, intraarticular injection and subcutaneous injection. On the other hand, no reaction is observed when BMP-7 is injected intravenously or into the intervertebral disk (OP-1 Immunogenicity Report, FDA StrykerBiotech Briefing for Mar. 31, 2009 Advisory Committee Meeting). These immunological reactions during the use of BMP-7 for regeneration applications are described in the article by C-J Hwang et al. (J Neurosurg Spine 13:484-493, 2010 and J Neurosurg 10:443-451, 2009). Patients who are thus treated with BMP-7 and who develop anti-BMP-7 antibodies that can neutralize the biological activity run the risk of experiencing a reduction in the efficacy of the treatment. Furthermore, these antibodies can potentially also react with endogenous BMP-7 and neutralize its activity, thus increasing the risk of side effects.

As regards BMP-2, the dissolving of BMP-2 lyophilisates and the stability of the injectable formulations have already been mentioned in application PCT/EP2008/059832. To date, the number of systemic applications of BMP-2 described in the literature is limited, but some have been mentioned, such as cardiac regeneration (Bone Morphogenetic Proteins: From local to systemic therapeutics, Eds S. Vukicevic and K. Sampath, 2008, Birkhaüser, p 317-337).

In addition, it appears to be necessary to obtain effective formulations containing a minimum amount of BMP-2 and BMP-7, in order to avoid the side effects generated by high concentrations of this protein and also owing to the cost of this protein.

One of the solutions for answering the problem of the low solubility of BMP-7 at neutral pH, developed by the company Centocor, consists in modifying the primary structure of BMP-7 (Swencki-Underwood, B. et al., Protein Expr. Purif. 2008, 57 (2), 312-319). However, this solution is not satisfactory since it results in a potential toxicity of the modified new protein and since it induces a modification of the interactions between BMP-7 and its receptors which may result in a modification of the biological activity.

Another proposed solution to the low solubility of BMP-7 at neutral pH, described in patent application US2007/0015701, consists in covalently grafting one or more polyethylene glycol chains onto BMP-7 (Zalipsky, Samuel et al., US2007/0015701 A1). This solution is also unsatisfactory since the BMP-7 is chemically modified, which can result in significant modifications to its biological activity compared with the natural protein.

The applicant had already described a solution in application PCT/EP2008/059832, making it possible to solve the similar problems of solubility at physiological pH with BMP-2 without having recourse to chemical modifications of BMP-2. This solution consisted in using an amphiphilic polysaccharide comprising a hydrophobic group chosen from the group consisting of hydrophobic amino acids of natural origin, chosen from the group consisting of tryptophan, tyrosine, phenylalanine, leucine or isoleucine, or alcohol, ester, decarboxylated or amide derivatives thereof.

Surprisingly, the applicant has demonstrated that some polysaccharides, in addition to the fact that they form complexes with BMP-2 and BMP-7, make it possible to solubilize these growth factors at physiological pH with a low polysaccharide/BMP mass ratio.

They also make it possible to reduce the immunogenicity of the BMP-7 formulations.

Furthermore, these complexes have the advantage of being stable under physiological conditions, but also with respect to considerable dilution in serum.

These polysaccharides also have the property of being lyoprotectant and make it possible to maintain the integrity of BMP-2 and of BMP-7 while avoiding aggregation phenomena during lyophilization processes.

### BRIEF DESCRIPTION OF THE DRAWINGS

FIG. 1 is an illustration of the anti-rhBMP-7 IgG titer observed with composition C1 according to the present disclosure.

FIG. 2 is an illustration of the anti-rhBMP-7 IgG titer observed with composition C2 according to the present disclosure.

### DETAILED DESCRIPTION OF EMBODIMENTS

The present invention relates to a polysaccharide/BMP complex, the BMP being chosen from the group consisting of BMP-2 and BMP-7, said complex being soluble at physiological pH, wherein the polysaccharide/BMP mass ratio is less than 15, the polysaccharide being chosen from the group of polysaccharides comprising carboxyl functional groups, at least one of which is substituted with at least one hydrophobic radical, denoted Ah:
said hydrophobic radical Ah being a residue of a hydrophobic compound chosen from hydrophobic alcohols or acids comprising a linear, branched or cyclic alkyl chain containing at least 6 carbon atoms, said hydrophobic radical Ah being bonded to a linker arm R by a function G resulting from coupling between at least one reactive function of said hydrophobic compound and a reactive function of the linker arm precursor R′,
said linker arm R being bonded to the polysaccharide by a bond F resulting from coupling between a reactive function of the linker arm precursor R′ and a carboxyl function of the anionic polysaccharide, R being an at least divalent radical consisting of a chain comprising between 1 and 15 carbons, which is optionally branched and/or unsaturated, optionally comprising one or more heteroatoms, such as O, N and/or S, and resulting from a precursor R′ having at least two reactive functions, at least one being an amine function and the others, which are identical or different, being chosen from the group consisting of alcohol, acid or amine functions,
F being an amide function,
G being either an amide, ester or carbamate function,
the unsubstituted carboxyl functions of the anionic polysaccharide being in the form of a cation carboxylate, preferably an alkali cation, preferably such as Na⁺ or K⁺,
said polysaccharide comprising carboxyl functional groups which are amphiphilic at neutral pH,
the BMP being chosen from the group consisting of recombinant human BMP-2 and BMP-7, and homologs thereof.

In one embodiment, the BMP is chosen from the group consisting of recombinant human BMP-2s and homologs thereof.

In one embodiment, the BMP is chosen from the group consisting of recombinant human BMP-7s and homologs thereof.

In one embodiment, the polysaccharide/BMP mass ratio is less than 10.

In one embodiment, the polysaccharide/BMP mass ratio is less than 5.

In one embodiment, the polysaccharide/BMP mass ratio is less than 3.

In one embodiment, the polysaccharides comprising carboxyl functional groups are synthetic polysaccharides obtained from neutral polysaccharides, onto which at least 15 carboxyl functional groups per 100 saccharide units have been grafted, of general formula I:

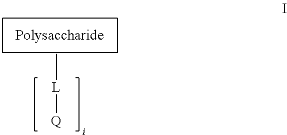

the natural polysaccharides being chosen from the group of polysaccharides of which the bonds between the glycosidic monomers comprise (1,6)-bonds,
L being a bond resulting from coupling between a precursor of the linker arm Q and an —OH function of the polysaccharide and being either an ester, carbamate or ether function,
i represents the molar fraction of the substituents L-Q per saccharide unit of the polysaccharide,
Q being chosen from the radicals of general formula II:

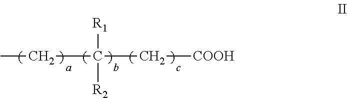

in which:

1≦a+b+c≦6, and

0≦a≦3,

0≦b≦3

0≦c≦3,

R₁ and R₂, which may be identical or different, are chosen from the group consisting of —H, linear or branched C₁ to C₃ alkyl, —COON and the radical of formula III in which

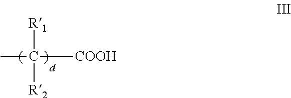

1≦d≦3, and

R′₁ and R′₂, which may be identical or different, are chosen from the group consisting of —H and a linear or branched C₁ to C₃ alkyl group.

In one embodiment, a+b+c≦5.

In one embodiment, a+b+c≦4.

In one embodiment, i is between 0.1 and 3.

In one embodiment, i is between 0.2 and 1.5.

In one embodiment, the polysaccharide is chosen from the group consisting of polysaccharides of which the bonds between the glycosidic monomers comprise (1,6)-bonds.

In one embodiment, the polysaccharide is chosen from the group consisting of dextran and pullulan.

In one embodiment, the polysaccharide chosen from the group consisting of polysaccharides of which the bonds between the glycosidic monomers comprise (1,6)-bonds is dextran.

In one embodiment, the polysaccharide is chosen from the group consisting of polysaccharides of which the bonds between the glycosidic monomers comprise (1,6)-bonds and (1,4)-bonds.

In one embodiment, the polysaccharide of which the bonds between the glycosidic monomers comprise (1,6 bonds and (1,4)-bonds is a pullulan.

In one embodiment, the polysaccharide according to the invention is characterized in that the L-Q radical is chosen from the group consisting of the following radicals, L having the meaning given above:

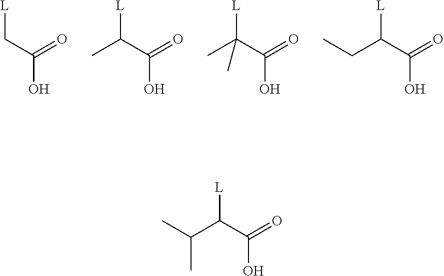

In one embodiment, the polysaccharide according to the invention is characterized in that the L-Q radical is chosen from the group consisting of the following radicals, L having the meaning given above:

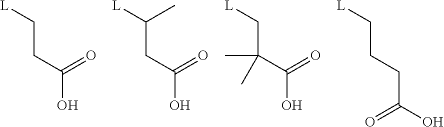

In one embodiment, the polysaccharide according to the invention is characterized in that the L-Q radical is chosen from the group consisting of the following radicals, L having the meaning given above:

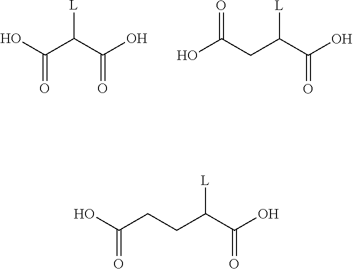

In one embodiment, the polysaccharide is chosen from the polysaccharides of formula (IV):

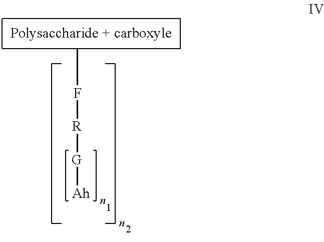

Ah being a residue of a hydrophobic compound chosen from hydrophobic alcohols or acids comprising a linear, branched or cyclic alkyl chain containing at least 6 carbon atoms, produced from coupling between a hydroxyl or acid function of the hydrophobic compound and a reactive function of the precursor R′ of R,
F being an amide function,
G being either an ester function, or a carbamate function, or an amide function,
R being an at least divalent radical consisting of a chain comprising between 1 and 15 carbons, which is optionally branched and/or unsaturated, optionally comprising one or more heteroatoms, such as O, N and/or S, resulting from a precursor R′ having at least two reactive functions, at least one being an amine function and the others, which are identical or different, being chosen from the group consisting of alcohol, acid or amine functions,
n₁ being equal to 1 or 2,
n₂ representing the molar fraction of the carboxyl functions of the polysaccharide which are substituted with F—R-G-Ah and being between 0.01 and 0.7, and
when the carboxyl function of the polysaccharide is not substituted with F—R-G-Ah, then the carboxy functional group(s) of the polysaccharide are cation carboxylates, preferably alkali cation, such as Na⁺ or K.

In one embodiment, n₂ is between 0.02 and 0.5.

In one embodiment, n₂ is between 0.05 and 0.3.

In one embodiment, n₂ is between 0.1 and 0.2.

In one embodiment, n₁ is equal to 1 and the precursor R′ of the group R comprises two reactive functions.

In one embodiment, F is an amide function, G is an ester function, R′ is an amino acid and Ah is a hydrophobic alcohol residue.

In one embodiment, F is an amide function, G is a carbamate function, R′ is a diamine and Ah is a hydrophobic alcohol residue.

In one embodiment, F is an amide function, G is an amide function, R′ is a diamine and Ah is a hydrophobic acid residue.

In one embodiment, the precursor R′ of the group R, comprising two reactive functions, is characterized in that it is chosen from amino acids.

In one embodiment, the amino acids are chosen from alpha-amino acids.

In one embodiment, the alpha-amino acids are chosen from natural alpha-amino acids.

In one embodiment, the natural alpha-amino acids are chosen from leucine, alanine, isoleucine, glycine, phenylalanine, valine, proline and aspartic acid.

In one embodiment, the precursor R′ of the group R, comprising two reactive functions, is characterized in that it is chosen from diamines.

In one embodiment, the diamines are chosen from the group consisting of ethylenediamine and lysine and its derivatives.

In one embodiment, the precursor R′ of the group R is characterized in that it is chosen from alcohol amines.

In one embodiment, the alcohol amines are chosen from the group consisting of ethanolamine, amino-2-propanol, isopropanolamine, 3-amino-1,2-propanediol, diethanolamine, diisopropanolamine, tromethamine (tris) and 2-(2-aminoethoxy)ethanol.

In one embodiment, the alcohol amines are chosen from the group consisting of reduced amino acids.

In one embodiment, the reduced amino acids are chosen from the group consisting of alaminol, valinol, leucinol, isoleucinol, prolinol and phenylalaminol.

In one embodiment, the alcohol amines are chosen from the group consisting of charged amino acids.

In one embodiment, the charged amino acids are chosen from the group consisting of serine and threonine.

In one embodiment, n₁ is equal to 2 and the percursor R′ of the group R comprises three reactive functions.

In one embodiment, the precursor R′ comprising three reactive functions is chosen from amino acids bearing two amine functions.

The amino acids bearing two amine functions are chosen from the group consisting of lysine, 5-hydroxylysine, 2,4-diaminobutyric acid, 2,3-diaminopropionic acid, ornithine and p-aminophenylalanine.

In one embodiment, the precursor R′ comprising at least three reactive functions is chosen from amino acids bearing an alcohol function.

The amino acids bearing an alcohol function are chosen from the group consisting of serine, threonine, tyrosine, homoserine and alpha-methylserine.

In one embodiment, the precursor R′ comprising three reactive functions is chosen from alcohol amines.

The alcohol amines are chosen from the group consisting of tromethamine (tris), 3-amino-1,2-propanediol, triethanolamine, hydroxymethyltyrosine, tyrosinol, serinol (2-amino-1,2-propanediol) and threoninol.

In one embodiment, the precursor R′ comprising three reactive functions is chosen from triamines.

In one embodiment, the triamines are chosen from the group consisting of 2-(aminomethyl)-2-methyl-1,3-propanediamine and tris(2-aminoethyl)amine.

In one embodiment, the hydrophobic alcohol is chosen from alcohols consisting of a branched or unbranched, saturated or unsaturated alkyl chain comprising from 6 to 18 carbons.

In one embodiment, the hydrophobic alcohol is chosen from alcohols consisting of a branched or unbranched, saturated or unsaturated alkyl chain comprising more than 18 carbons.

In one embodiment, the hydrophobic alcohol is octanol.

In one embodiment, the hydrophobic alcohol is dodecanol.

In one embodiment, the hydrophobic alcohol is 2-ethylbutanol.

In one embodiment, the fatty alcohol is chosen from meristyl, cetyl, stearyl, cetearyl, butyl and oleyl alcohol and lanolin.

In one embodiment, the hydrophobic alcohol is chosen from cholesterol derivatives.

In one embodiment, the cholesterol derivative is cholesterol.

In one embodiment, the hydrophobic alcohol is chosen from menthol derivatives.

In one embodiment, the hydrophobic alcohol is menthol in the racemic form thereof.

In one embodiment, the hydrophobic alcohol is the D isomer of menthol.

In one embodiment, the hydrophobic alcohol is the L isomer of menthol.

In one embodiment, the hydrophobic alcohol is chosen from tocopherols.

In one embodiment, the tocopherol is alpha-tocopherol.

In one embodiment, the alpha-tocopherol is the racemate of alpha-tocopherol.

In one embodiment, the tocopherol is the D isomer of alpha-tocopherol.

In one embodiment, the tocopherol is the L isomer of alpha-tocopherol.

In one embodiment, the hydrophobic alcohol is chosen from alcohols bearing an aryl group.

In one embodiment, the alcohol bearing an aryl group is chosen from benzyl alcohol and phenethyl alcohol.

In one embodiment, the hydrophobic alcohol is chosen from the unsaturated fatty alcohols in the group consisting of geraniol, β-citronellol and farnesol.

In one embodiment, the hydrophobic alcohol is 3,7-dimethyl-1-octanol.

In one embodiment, the hydrophobic acid is chosen from fatty acids.

In one embodiment, the fatty acids are chosen from the group consisting of acids consisting of a branched or unbranched, saturated or unsaturated alkyl chain comprising from 6 to 50 carbons.

In one embodiment, the fatty acids are chosen from the group consisting of linear fatty acids.

In one embodiment, the linear fatty acids are chosen from the group consisting of caproic acid, enanthic acid, caprylic acid, capric acid, nonanoic acid, decanoic acid, undecanoic acid, dodecanoic acid, palmitic acid, stearic acid, arachidic acid, behenic acid, tricosanoic acid, lignoceric acid, heptacosanoic acid, octacosanoic acid and melissic acid.

In one embodiment, the fatty acids are chosen from the group consisting of unsaturated fatty acids.

In one embodiment, the unsaturated fatty acids are chosen from the group consisting of myristoleic acid, palmitoleic acid, oleic acid, elaidic acid, linoleic acid, alpha-linoleic acid, arachidonic acid, eicosapentaenoic acid, erucic acid and docosahexaenoic acid.

In one embodiment, the fatty acids are chosen from the group consisting of bile acids and derivatives thereof.

In one embodiment, the bile acids and derivatives thereof are chosen from the group consisting of cholic acid, dehydrocholic acid, deoxycholic acid and chenodeoxycholic acid.

In one embodiment, the invention relates to a polysaccharide/BMP-2 complex chosen from the group consisting of the following complexes:
40 kDa sodium dextranmethylcarboxylate modified with octanol phenylalaninate/BMP-2, mass ratio=10.
40 kDa sodium dextranmethylcarboxylate modified with dihexanol aspartate/BMP-2, mass ratio=10.
40 kDa dextranmethylcarboxylate modified with N-(2-aminoethyl)dodecanamide/BMP-2, mass ratio=10.
40 kDa dextranmethylcarboxylate modified with octanol leucinate, mass ratio=10.
40 kDa sodium dextranmethylcarboxylate modified with dodecanol glycinate, mass ratio=10.
40 kDa sodium dextranmethylcarboxylate modified with octanol glycinate/BMP-2, mass ratio=6.25.
40 kDa dextranmethylcarboxylate modified with octanol phenylalaninate/BMP-2, mass ratio=6.25.
40 kDa dextranmethylcarboxylate modified with dodecanol alaninate/BMP-2, mass ratio=6.25.

In one embodiment, the invention relates to a polysaccharide/BMP-7 complex chosen from the group consisting of the following complexes:
40 kDa sodium dextranmethylcarboxylate modified with octanol glycinate/BMP-7, mass ratio=10.
40 kDa sodium dextranmethylcarboxylate modified with octanol glycinate/BMP-7, mass ratio=12.3.
10 kDa sodium dextranmethylcarboxylate modified with octanol glycinate/BMP-7, mass ratio=10.
10 kDa sodium dextranmethylcarboxylate modified with octanol glycinate/BMP-7, mass ratio=4.
10 kDa sodium dextranmethylcarboxylate modified with dodecanol glycinate/BMP-7, mass ratio=10.
10 kDa sodium dextranmethylcarboxylate modified with isohexanol leucinate/BMP-7, mass ratio=10.
40 kDa sodium dextranmethylcarboxylate modified with octanol phenylalaninate/BMP-7, mass ratio=10.
40 kDa sodium dextranmethylcarboxylate modified with octanol phenylalaninate/BMP-7, mass ratio=4.
40 kDa sodium dextranmethylcarboxylate modified with octanol valinate/BMP-7, mass ratio=10.
40 kDa sodium dextranmethylcarboxylate modified with ethanolamine laurate ester/BMP-7, mass ratio=10.
40 kDa sodium dextranmethylcarboxylate modified with dihexanol aspartate/BMP-7, mass ratio=10.
10 kDa dextranmethylcarboxylate modified with cholesterol leucinate/BMP-7, mass ratio=10.
10 kDa dextranmethylcarboxylate modified with octanol phenylalaninate/BMP-7, mass ratio=10.
10 kDa dextranmethylcarboxylate modified with 3,7-dimethyl-1-octanol phenylalaninate/BMP-7, mass ratio=10.
10 kDa dextranmethylcarboxylate modified with 2-(2-aminoethoxy)ethyl octanoate/BMP-7, mass ratio=10.
10 kDa dextranmethylcarboxylate modified with 2-(2-aminoethoxy)ethyl dodecanoate/BMP-7, mass ratio=10.
10 kDa dextranmethylcarboxylate modified with N-[2-((2-octanoylamino-3-phenyl)propanoylamino)]ethanamine/BMP-7, mass ratio=10.
10 kDa dextranmethylcarboxylate modified with N-[2-((2-octanoylamino-3-phenyl)propanoylamino)]ethanamine/BMP-7, mass ratio=4.
10 kDa dextranmethylcarboxylate modified with N-(2-aminoethyl)octanamide/BMP-7, mass ratio=10.
40 kDa dextranmethylcarboxylate modified with N-(2-aminoethyl)dodecanamide/BMP-7, mass ratio=10.
40 kDa dextranmethylcarboxylate modified with N-(2-aminoethyl)dodecanamide/BMP-7, mass ratio=4.
10 kDa sodium dextranmethylcarboxylate modified with didodecanol aspartate/BMP-7, mass ratio=10.
10 kDa dextran carbamate N-methyl(sodium carboxylate) modified with N-(2-aminoethyl)dodecanamide/BMP-7, mass ratio=4.
10 kDa dextranmethylcarboxylate modified with isohexanol phenylalaninate/BMP-7, mass ratio=10.
10 kDa dextranmethylcarboxylate modified with benzyl phenylalaninate/BMP-7, mass ratio=10.
10 kDa dextranmethylcarboxylate modified with isohexanol phenylalaninate/BMP-7, mass ratio=10.

The invention also relates to a therapeutic composition, which comprises an amphiphilic polysaccharide/BMP-7 complex according to the invention.

The invention also relates to a therapeutic composition, which comprises an amphiphilic polysaccharide/BMP-2 complex according to the invention.

The term “therapeutic composition” is intended to mean a composition that can be used in human or veterinary medicine.

In one embodiment, the pharmaceutical composition according to the invention is a locally applied composition which may be in the form of a solute, a gel, a cream, a lyophilisate, a powder or a paste.

In one embodiment, the pharmaceutical composition according to the invention is a systemically applied composition for intravenous or subcutaneous administration, which may be in the form of a solute.

The nature of the excipients which can be formulated with the amphiphilic polysaccharide/BMP complex according to the invention is chosen according to the presentation form thereof, according to the general knowledge of the specialist in galenical pharmacology.

Thus, when the composition according to the invention is in the form of a paste or of a cement, it is, for example, obtained from products such as carboxymethylcelluloses (CMCs), tricalcium phosphate and collagen.

Other excipients can be used in this invention in order to adjust the parameters of the formulation, such as a buffer for adjusting the pH, an agent for adjusting the isotonicity, preservatives such as methyl para-hydroxybenzoate, propyl para-hydroxybenzoate, m-cresol or phenol, or else an antioxidant agent such as L-lysine hydrochloride.

According to the invention, the therapeutic composition is characterized in that it allows an administration of approximately 10 mg/ml of BMP-7 or of BMP-2.

According to the invention, the therapeutic composition is characterized in that it allows an administration of approximately 5 mg/ml of BMP-7 or of BMP-2.

According to the invention, the therapeutic composition is characterized in that it allows an administration of approximately 2 mg/ml of BMP-7 or of BMP-2.

According to the invention, the therapeutic composition is characterized in that it allows an administration of approximately 1 mg/ml of BMP-7 or of BMP-2.

According to the invention, the therapeutic composition is characterized in that it allows an administration of approximately 0.2 mg/ml of BMP-7 or of BMP-2.

The present invention also relates to the use of an amphiphilic polysaccharide/BMP-7 or amphiphilic polysaccharide/BMP-2 complex according to the invention, for the preparation of a therapeutic composition for use in inducing bone formation in vivo.

The present invention also relates to the use of an amphiphilic polysaccharide/BMP-7 or amphiphilic polysaccharide/BMP-2 complex according to the invention, for the preparation of a therapeutic composition for use in inducing cartilage regeneration.

The present invention also relates to the use of an amphiphilic polysaccharide/BMP-7 complex according to the invention, for the preparation of a therapeutic composition for use in inducing kidney regeneration.

The present invention also relates to the use of an amphiphilic polysaccharide/BMP-7 complex according to the invention, for the preparation of a therapeutic composition for use in inducing liver regeneration.

The present invention also relates to the use of an amphiphilic polysaccharide/BMP-7 complex according to the invention, for the preparation of a therapeutic composition for use in inducing corneal regeneration.

The present invention also relates to the use of an amphiphilic polysaccharide/BMP-7 complex according to the invention, for the preparation of a therapeutic composition for use in treating strokes.

The present invention also relates to the use of an amphiphilic polysaccharide/BMP-7 complex according to the invention, for the preparation of a therapeutic composition for use in treating myocardial infarction.

The present invention also relates to the use of an amphiphilic polysaccharide/BMP-7 complex according to the invention, for the preparation of a therapeutic composition for use in treating peripheral arterial diseases.

The present invention also relates to the use of an amphiphilic polysaccharide/BMP-7 complex according to the invention, for the preparation of a therapeutic composition for use in treating chronic obstructive pulmonary diseases.

The present invention also relates to the use of an amphiphilic polysaccharide/BMP-7 complex according to the invention, for the preparation of a therapeutic composition for use in treating critical lower limb ischemia.

It also relates to a method of therapeutic treatment for human or veterinary use, which consists in administering, at the site of treatment, a therapeutic composition comprising the amphiphilic polysaccharide/BMP-7 or amphiphilic polysaccharide/BMP-2 complex according to the invention.

It also relates to a method of therapeutic treatment for human or veterinary use, which consists in intravenously administering a therapeutic composition comprising the amphiphilic polysaccharide/BMP-7 complex according to the invention.

It also relates to a method of therapeutic treatment for human or veterinary use, which consists in subcutaneously administering a therapeutic composition comprising the amphiphilic polysaccharide/BMP-7 complex according to the invention.

The pharmaceutical compositions according to the invention are either in liquid form, in an aqueous solution, or in powder form, or in the form of a lyophilisate, an implant or a film. They also comprise the conventional pharmaceutical excipients well known to those skilled in the art.

Depending on the pathologiescal conditions and the methods modes of administration, the pharmaceutical compositions may advantageously also comprise excipients which make it possible to formulate them in the form of a gel, a sponge, an injectable solution, an oral solution, a lyoc, etc.

The invention also relates to a pharmaceutical composition according to the invention as described above, which can be administered in the form of a stent, of an implantable biomaterial film or coating or of an implant.

In one embodiment, the invention relates to a liquid pharmaceutical formulation containing BMP-7 at 1 mg/ml at physiological pH, the composition of which is the following:

polysaccharide/BMP-7 complex of mass ratio 4 corresponding to 1 mg/ml of BMP-7,
10 mM of phosphate buffer,
7.1% of trehalose (osmotic agent).

### Example 1

### Synthesis of Sodium Dextranmethylcarboxylate Modified with Octanol Glycinate

### Polymer 1

The octanol glycinate, para-toluenesulfonic acid salt, is obtained according to the process described in the patent (M Kenji et al., U.S. Pat. No. 4,826,818).

8 g (i.e. 148 mmol of hydroxyl functions) of dextran having a weight-average molar mass of approximately 40 kg/mol (Fluka) are solubilized in water at 42 g/l. 15 ml of 10N NaOH (148 mmol NaOH) are added to this solution. The mixture is brought to 35° C. and then 23 g (198 mmol) of sodium chloroacetate are added. The temperature of the reaction medium is brought to 60° C. at 0.5° C./min and then maintained at 60° C. for 100 minutes. The reaction medium is diluted with 200 ml of water, neutralized with acetic acid and purified by ultrafiltration through a 5 kD PES membrane against 6 volumes of water. The final solution is assayed by dry extract to determine the polymer concentration; and then assayed by acid/base titration in 50/50 (v/v) water/acetone to determine the degree of substitution with methylcarboxylates.

According to the dry extract: [polymer]=31.5 mg/g.

According to the acid/base titration: the degree of substitution of the hydroxyl functions with methylcarboxylate functions is 1.04 per saccharide unit.

The sodium dextranmethylcarboxylate solution is passed through a Purolite resin (anionic) to obtain dextranmethylcarboxylic acid, which is then lyophilized for 18 hours.

8 g of dextranmethylcarboxylic acid (37 mmol of methylcarboxylic acid functions) are solubilized in DMF at 78 g/L and then cooled to 0° C. 2.59 g of octanol glycinate, para-toluenesulfonic acid salt (7.2 mmol) are suspended in DMF at 100 g/L. 0.73 g of triethylamine (7.2 mmol) is then added to this suspension. Once the solution of polymer is at 0° C., 4.16 g (41 mmol) of NMM and 4.47 g (41 mmol) of EtOCOCl are then added. After 10 min of reaction, the octanol glycinate solution is added and the medium is maintained at 10° C. for 45 minutes. The medium is then heated to 50° C. At 30° C., an aqueous solution of imidazole at 600 g/L and 40 mL of water are added. After 1 h 30 of stirring at 50° C., the solution obtained is ultrafiltered through a 10 kD PES membrane against 6 volumes of 0.9% NaCl solution, 3 volumes of 0.01N sodium hydroxide, 8 volumes of 0.9% NaCl solution and then 3 volumes of water. The concentration of the polymer solution is determine by dry extract. A fraction of the solution is lyophilized and analyzed by ¹H NMR in D₂O to determine the rate of acid functions converted to amide of octanol glycinate.

According to the dry extract: [polymer 1]=30.2 mg/g.

According to the ¹H NMR: the molar fraction of acids modified with octanol glycinate per saccharide unit is 0.21.

### Example 2

### Synthesis of Sodium Dextranmethylcarboxylate Modified with Octanol Glycinate

### Polymer 2

The octanol glycinate, para-toluenesulfonic acid salt, is obtained according to the process described in the patent (M Kenji et al., U.S. Pat. No. 4,826,818).

A sodium dextranmethylcarboxylate modified with octanol glycinate is obtained by means of a process similar to that described in example 1, starting from a dextran having a weight-average molar mass of 10 kDa.

According to the dry extract: [polymer 2]=30.6 mg/g.

According to the ¹H NMR: the molar fraction of acids modified with octanol glycinate per saccharide unit is 0.16.

### Example 3

### Synthesis of Sodium Dextranmethylcarboxylate Modified with Dodecanol Glycinate

### Polymer 3

The dodecanol glycinate, para-toluenesulfonic acid salt, is obtained according to the process described in the patent (M Kenji et al., U.S. Pat. No. 4,826,818).

A sodium dextranmethylcarboxylate modified with dodecanol glycinate is obtained by means of a process similar to that described in example 1, starting from the dextran having a weight-average molar mass of 10 kDa.

According to the dry extract: [polymer 3]=23.6 mg/g.

According to the ¹H NMR: the molarfraction of acids modified with dodecanol glycinate per saccharide unit is 0.10.

### Example 4

### Synthesis of Sodium Dextranmethylcarboxylate Modified with Isohexanol Leucinate

### Polymer 4

The isohexanol leucinate, para-toluenesulfonic acid salt, is obtained according to the process described in the patent (M Kenji et al., U.S. Pat. No. 4,826,818).

A sodium dextranmethylcarboxylate modified with isohexanol leucinate is obtained by means of a process similar to that described in example 1, starting from a dextran having a weight-average molar mass of 10 kDa.

According to the dry extract: [polymer 4]=12.3 mg/g.

According to the ¹H NMR: the molar fraction of acids modified with isohexanol leucinate per saccharide unit is 0.18.

### Example 5

### Synthesis of Sodium Dextranmethylcarboxylate Modified with Octanol Phenylalaninate

### Polymer 5

The octanol phenylalaninate, para-toluenesulfonic acid salt, is obtained according to the process described in the patent (M Kenji et al., U.S. Pat. No. 4,826,818).

A sodium dextranmethylcarboxylate modified with octanol phenylalaninate is obtained by means of a process similar to that described in example 1, starting from a dextran having a weight-average molar mass of 40 kDa.

According to the dry extract: [polymer 5]=30.2 mg/g.

According to the ¹H NMR: the molar fraction of acids modified with octanol phenylalaninate per saccharide unit is 0.10.

### Example 6

### Synthesis of Sodium Dextran Succinate Modified with Octanol Glycinate

### Polymer 6

The octanol glycinate, para-toluenesulfonic acid salt, is obtained according to the process described in the patent (M Kenji et al., U.S. Pat. No. 4,826,818).

A sodium dextran succinate is obtained starting from a dextran having a weight-average molar mass of 10 kDa (Pharmacosmos) according to the method described in the article by Sanchez Chaves et al. (Sanchez Chaves, Manuel et al., Polymer 1998, 39(13), 2751-2757). The rate of acid functions per glycosidic unit is 1.4 according to the ¹H NMR in NaOD/D₂O.

A sodium dextran succinate modified with octanol glycinate is obtained by means of a process similar to that described in example 1.

According to the dry extract: [polymer 6]=23.6 mg/g.

According to the ¹H NMR: the molar fraction of acids modified with doodecanol glycinate per saccharide unit is 0.10.

### Example 7

### Synthesis of Sodium Dextranmethylcarboxylate Modified with Octanol Valinate

### Polymer 7

The octanol valinate, para-toluenesulfonic acid salt, is obtained according to the process described in the patent (M Kenji et al., U.S. Pat. No. 4,826,818).

A sodium dextranmethylcarboxylate modified with octanol valinate is obtained by means of a process similar to that described in example 1, starting from a dextran having a weight-average molar mass of 40 kDa.

According to the dry extract: [polymer 7]=33.2 mg/g.

According to the ¹H NMR: the molar fraction of acids modified with octanol valinate per saccharide unit is 0.08.

### Example 8

### Synthesis of Sodium Dextranmethylcarboxylate Modified with Ethanolamine Laurate Ester

### Polymer 8

The ethanolamine laurate ester, para-toluenesulfonic acid salt, is obtained according to the process described in the patent (M Kenji et al., U.S. Pat. No. 4,826,818).

A sodium dextranmethylcarboxylate modified with ethanolamine laurate ester is obtained by means of a process similar to that described in example 1, starting from a dextran having a weight-average molar mass of 40 kDa.

According to the dry extract: [polymer 8]=21.2 mg/g.

According to the ¹H NMR: the molar fraction of acids modified with ethanolamine laurate ester per saccharide unit is 0.09.

### Example 9

### Synthesis of Sodium Dextranmethylcarboxylate Modified with Dihexanol Aspartate

### Polymer 9

The dihexanol aspartate, para-toluenesulfonic acid salt, is obtained according to the process described in the patent (M Kenji et al., U.S. Pat. No. 4,826,818).

A sodium dextranmethylcarboxylate modified with dihexanol aspartate is obtained by means of a process similar to that described in example 1, starting from a dextran having a weight-average molar mass of 40 kDa.

According to the dry extract: [polymer 9]=31.1 mg/g.

According to the ¹H NMR: the molar fraction of acids modified with dihexanol aspartate per saccharide unit is 0.075.

### Example 10

### Synthesis of Sodium Dextranmethylcarboxylate Modified with Dodecanol Glycinate

### Polymer 10

The dodecanol glycinate, para-toluenesulfonic acid salt, is obtained according to the process described in the patent (M Kenji et al., U.S. Pat. No. 4,826,818).

A sodium dextranmethylcarboxylate modified with dodecanol glycinate is obtained by means of a process similar to that described in example 1, starting from a dextran having a weight-average molar mass of 40 kDa.

According to the dry extract: [polymer 10]=25.3 mg/g.

According to the ¹H NMR: the molar fraction of acids modified with dodecanol glycinate per saccharide unit is 0.1.

### Example 11

### Synthesis of Sodium Dextranmethylcarboxylate Modified with Octanol Leucinate

### Polymer 11

The octanol leucinate, para-toluenesulfonic acid salt, is obtained according to the process described in the patent (M Kenji et al., U.S. Pat. No. 4,826,818).

A sodium dextranmethylcarboxylate modified with octanol leucinate is obtained by means of a process similar to that described in example 1, starting from a dextran having a weight-average molar mass of 40 kDa.

According to the dry extract: [polymer 11]=32.9 mg/g.

According to the ¹H NMR: the molar fraction of acids modified with octanol leucinate per saccharide unit is 0.10.

### Example 12

### Synthesis of Dextranmethylcarboxylate Modified with Cholesterol Leucinate

### Polymer 12

The cholesterol leucinate, para-toluenesulfonic acid salt, is obtained according to the process described in the patent (M Kenji et al., U.S. Pat. No. 4,826,818).

A sodium dextranmethylcarboxylate modified with cholesterol leucinate is obtained by means of a process similar to that described in example 1, starting from a dextran having a weight-average molar mass of 10 kDa.

According to the dry extract: [polymer 12]=25.8 mg/g.

According to the ¹H NMR: the molar fraction of acids modified with cholesterol leucinate per saccharide unit is 0.03.

### Example 13

### Synthesis of Dextranmethylcarboxylate Modified with Octanol Phenylalaninate

### Polymer 13

The octanol phenylalaninate, para-toluenesulfonic acid salt, is obtained according to the process described in the patent (M Kenji et al., U.S. Pat. No. 4,826,818).

A sodium dextranmethylcarboxylate modified with octanol phenylalaninate is obtained by means of a process similar to that described in example 1, starting from a dextran having a weight-average molar mass of 10 kDa.

According to the dry extract: [polymer 13]=36.9 mg/g.

According to the ¹H NMR: the molar fraction of acids modified with octanol phenylalaninate per saccharide unit is 0.2.

### Example 14

### Synthesis of Dextranmethylcarboxylate Modified with 3,7-dimethyl-1-octanol phenylalaninate

### Polymer 14

The 3,7-dimethyl-1-octanol phenylalaninate, para-toluenesulfonic acid salt, is obtained according to the process described in the patent (M Kenji et al., U.S. Pat. No. 4,826,818).

A sodium dextranmethylcarboxylate modified with 3,7-dimethyl-1-octanol phenylalaninate is obtained by means of a process similar to that described in example 1, starting from a dextran having a weight-average molar mass of 10 kDa.

According to the dry extract: [polymer 14]=24.3 mg/g.

According to the ¹H NMR: the molar fraction of acids modified with 3,7-dimethyl-1-octanol phenylalaninate per saccharide unit is 0.1.

### Example 15

### Synthesis of Dextranmethylcarboxylate Modified with 2-(2-aminoethoxy)ethyl octanoate

### Polymer 15

The 2-(2-aminoethoxy)ethyl octanoate, para-toluenesulfonic acid salt, is obtained according to the process described in the patent (M Kenji et al., U.S. Pat. No. 4,826,818).

A sodium dextranmethylcarboxylate modified with 2-(2-aminoethoxy)ethyl octanoate is obtained by means of a process similar to that described in example 1, starting from a dextran having a weight-average molar mass of 10 kDa.

According to the dry extract: [polymer 15]=20.3 mg/g.

According to the ¹H NMR: the molar fraction of acids modified with 2-(2-aminoethoxy)ethyl octanoate per saccharide unit is 0.2.

### Example 16

### Synthesis of Dextranmethylcarboxylate Modified with 2-(2-aminoethoxy)ethyl dodecanoate

### Polymer 16

The 2-(2-aminoethoxy)ethyl dodecanoate, para-toluenesulfonic acid salt, is obtained according to the process described in the patent (M Kenji et al., U.S. Pat. No. 4,826,818).

A sodium dextranmethylcarboxylate modified with 2-(2-aminoethoxy)ethyl dodecanoate is obtained by means of a process similar to that described in example 1, starting from a dextran having a weight-average molar mass of 10 kDa.

According to the dry extract: [polymer 16]=25.6 mg/g.

According to the ¹H NMR: the molar fraction of acids modified with 2-(2-aminoethoxy)ethyl dodecanoate per saccharide unit is 0.1.

### Example 17

### Synthesis of Dextranmethylcarboxylate Modified with N-[2-((2-octanoylamino-3-phenyl)propanoylamino)]ethanamine

### Polymer 17

N-Octanoylphenylalanine is obtained according to the process described in the publication (A Pal et al., Tetrahedron 2007, 63, 7334-7348), starting from L-phenylalanine ethyl ester, hydrochloric acid salt (Bachem), and caprylic acid (Sigma).

The N-[2-((2-octanoylamino-3-phenyl)propanoylamino)]ethanamine, hydrochloric acid salt, is obtained according to the processes described in the publications (R Paul et al., J. Org. Chem. 1962, 27, 2094-2099 and D. J. Dale et al., Org. Process. Res. Dev. 2002, 6, 767-772), starting from N-octanoylphenylalanine and ethylenediamine (Roth).

A sodium dextranmethylcarboxylate modified with N-[2-((2-octanoylamino-3-phenyl)propanoylamino)]ethanamine is obtained by means of a process similar to that described in example 1, starting from a dextran having a weight-average molar mass of 10 kDa.

According to the dry extract: [polymer 17]=19.9 mg/g.

According to the ¹H NMR: the molar fraction of acids modified with N-[2-((2-octanoylamino-3-phenyl)propanoylamino)]ethanamine per saccharide unit is 0.1.

### Example 18

### Synthesis of Dextranmethylcarboxylate Modified with N-(2-aminoethyl)octanamide

### Polymer 18

The N-(2-aminoethyl)octanamide is obtained according to the process described in U.S. Pat. No. 2,387,201 (1945), starting from ethylenediamine (Roth) and caprylic acid (Sigma).

A sodium dextranmethylcarboxylate modified with N-(2-aminoethyl)octanamide is obtained by means of a process similar to that described in example 1, starting from a dextran having a weight-average molar mass of 10 kDa.

According to the dry extract: [polymer 18]=24.8 mg/g.

According to the ¹H NMR: the molar fraction of acids modified with N-(2-aminoethyl)octanamide per saccharide unit is 0.2.

### Example 19

### Synthesis of Dextranmethylcarboxylate Modified with N-(2-aminoethyl)dodecanamide

### Polymer 19

The N-(2-aminoethyl)dodecanamide is obtained according to the process described in U.S. Pat. No. 2,387,201 (1945), starting from ethylenediamine (Roth) and dodecanoic acid (Sigma).

A sodium dextranmethylcarboxylate modified with N-(2-aminoethyl)dodecanamide is obtained by means of a process similar to that described in example 1, starting from a dextran having a weight-average molar mass of 40 kDa.

According to the dry extract: [polymer 19]=15.7 mg/g.

According to the ¹H NMR: the molar fraction of acids modified with N-(2-aminoethyl)dodecanamide per saccharide unit is 0.1.

### Example 20

### Synthesis of Sodium Dextranmethylcarboxylate Modified with Didodecanol Aspartate

### Polymer 20

The didodecanol aspartate, para-toluenesulfonic acid salt, is obtained according to the process described in the patent (M Kenji et al., U.S. Pat. No. 4,826,818).

A sodium dextranmethylcarboxylate modified with didodecanol aspartate is obtained by means of a process similar to that described in example 1, starting from a dextran having a weight-average molar mass of 10 kDa.

According to the dry extract: [polymer 20]=20 mg/g.

According to the ¹H NMR: the molar fraction of acids modified with didodecanol aspartate per saccharide unit is 0.05.

### Example 21

### Synthesis of Dextran Carbamate N-methyl(sodium carboxylate) modified with N-(2-aminoethyl)dodecanamide

### Polymer 21

The N-(2-aminoethyl)dodecanamide is obtained according to the process described in U.S. Pat. No. 2,387,201 (1945).

11.5 g (i.e. 0.21 mol of hydroxyl functions) of dextran having a weight-average molar mass of approximately 10 kg/mol (Bachem) are solubilized in a DMF/DMSO mixture. The mixture is brought to 130° C. with stirring, and 13.75 g (0.11 mol) of ethyl isocyanatoacetate are gradually introduced. After 1 h of reaction, the medium is diluted in water and purified by difiltration through a 5 kD PES membrane against 0.1N NaOH, 0.9% NaCl and water. The final solution is assayed by dry extract to determine the polymer concentration; and then assayed by acid/base titration in 50/50 (v/v) water/acetone to determine the degree of substitution with carboxylate charges.

According to the dry extract: [polymer]=38.9 mg/g.

According to the acid/base titration: the degree of substitution of the hydroxyl functions with carbamate N-methylcarboxylate functions is 1.08 per saccharide unit.

The dextran carbamate N-methyl(sodium carboxylate) solution is passed through a Purolite resin (anionic) to obtain dextran carbamate N-methylcarboxylic acid, which is then lyophilized for 18 hours.

5 g of dextran carbamate N-methylcarboxylic acid are solubilized in DMF at 50 g/L and then cooled to 0° C. 2.22 g (22 mmol) of NMM and 2.38 g (22 mmol) of EtOCOCl are then added. After 10 min of reaction, 0.45 g (1.8 mmol) of N-(2-aminoethyl)dodecanamide is added and the medium is maintained at 10° C. for 45 minutes. The medium is then heated to 50° C. At 30° C., an aqueous solution of imidazole at 600 g/L and 25 mL of water are added. After 1 h 30 of stirring at 50° C., the solution obtained is ultrafiltered through a 10 kD PES membrane against 0.1N NaOH, 0.9% NaCl and water. The concentration of the polymer solution is determined by dry extract. A fraction of the solution is lyophilized and analyzed by ¹H NMR in D₂O to determine the rate of acid functions converted to amide of N-(2-aminoethyl)dodecanamide.

According to the dry extract: [polymer 21]=17.8 mg/g.

According to the ¹H NMR: the molar fraction of acids modified with N-(2-aminoethyl)dodecanamide per saccharide unit is 0.1.

### Example 22

### Synthesis of Dextranmethylcarboxylate Modified with Isohexanol Phenylalaninate

### Polymer 22

The isohexanol phenylalaninate, para-toluenesulfonic acid salt, is obtained according to the process described in the patent (M Kenji et al., U.S. Pat. No. 4,826,818).

A sodium dextranmethylcarboxylate modified with isohexanol phenylalaninate is obtained by means of a process similar to that described in example 1, starting from a dextran having a weight-average molar mass of 10 kDa.

According to the dry extract: [polymer 22]=28.1 mg/g.

According to the ¹H NMR: the molar fraction of acids modified with isohexanol phenylalaninate per saccharide unit is 0.2.

### Example 23

### Synthesis of Dextranmethylcarboxylate Modified with Benzyl Phenylalaninate

### Polymer 23

A sodium dextranmethylcarboxylate modified with benzyl phenylalaninate is obtained by means of a process similar to that described in example 1, starting from a dextran having a weight-average molar mass of 10 kDa, using benzyl phenylalaninate, hydrochloric acid salt (Bachem).

According to the dry extract: [polymer 23]=47.7 mg/g.

According to the ¹H NMR: the molar fraction of acids modified with benzyl phenylalaninate per saccharide unit is 0.45.

### Example 24

### Counterexample 1, Synthesis of Dextranmethylcarboxylate not Modified with a Hydrophobic Group

### Polymer 24

The sodium dextranmethylcarboxylate is obtained as described in the first part of example 1, starting from a dextran having a weight-average molar mass of 40 kDa. The mole fraction of acids modified with a hydrophobic group is zero.

### Example 25

### Synthesis of Sodium Dextranmethylcarboxylate Modified with Dodecanol Alaninate

### Polymer 25

The dodecanol alaninate, para-toluenesulfonic acid salt, is obtained according to the process described in the patent (M Kenji et al., U.S. Pat. No. 4,826,818).

A sodium dextranmethylcarboxylate solution obtained as described in example 1 is passed through a Purolite resin (anionic) to obtain dextranmethylcarboxylic acid, which is then lyophilized for 18 hours.

5 g of dextranmethylcarboxylic acid (23.2 mmol of methylcarboxylic acid functions) are solubilized in DMF at 45 g/L and then cooled to 0° C. 1.99 g of dodecanol alaninate, para-toluenesulfonic acid salt (4.6 mmol), is suspended in DMF at 100 g/L. 0.47 g of triethylamine (4.6 mmol) is then added to this suspension. Once the polymer solution is at 0° C., 2.35 g (23.2 mmol) of NMM and 2.52 g (23.2 mmol) of EtOCOCl are then added. After 10 min of reaction, the dodecanol alaninate suspension is added. The medium is then maintained at 4° C. for 15 minutes. The medium is then heated to 30° C. Once at 30° C., a solution of imidazole (3.2 g in 9.3 mL of water) is added to the reaction medium. The polymer solution is ultrafiltered through a 10 kD PES membrane against 10 volumes of 0.9% NaCl solution and then 5 volumes of water. The concentration of the polymer solution is determined by dry extract. A fraction of the solution is lyophilized and analyzed by ¹H NMR in D₂O to determine the rate of acid functions modified with dodecanol alaninate.

According to the dry extract: [modified polymer]=22 mg/g.

According to the ¹H NMR: the molar fraction of acids modified with dodecanol alaninate per saccharide unit is 0.19.

### Example 26

### Affinity of BMP-7 for a Polymer by Coelectrophoresis

Preparation of the BMP-7/Polymer Complex

5 μl of a solution of BMP-7 at 0.5 mg/ml are added to 2.5 μl of a solution of polymer (Pol) at 10 mg/ml and to 10 μl of 10× migration buffer (tris acetate, pH 7). This solution is made up to 100 μl with a solution of H₂O. This solution has a BMP-7 concentration of 25 μg/ml and a BMP-7/Pol ratio of 1/10.

Demonstration of the BMP-7/Polymer Complex

2 μl the BMP-7/Pol solution are then added to 8 μl of water and 2 μl of 5× loading buffer (glycerol, tris acetate and bromophenol blue in water). These 12 μl containing 50 ng of BMP-7 and 500 ng of polymer are loaded into a well of a 0.8% agarose gel. The electrophoresis tank is closed and the generator is set to 30V. The migration lasts for 1 hour.

After migration, the gel is transferred onto a PVDF membrane placed in a transfer apparatus, by capillarity for 2 h at room temperature (Apelex system). The membrane is saturated with PBST containing 5% of BSA for 45 minutes at room temperature and then incubated with primary BMP-7 antibodies (overnight at 4° C.) and, finally, incubated with rabbit anti-goat HRP-conjugated secondary antibodies (1 hour at room temperature). The developing is carried out by reaction of the HRP with Opti-4CN. The developing is stopped when the color is sufficient, since the product of the reaction absorbs in the visible range.

When the BMP-7 forms a complex with the polymer, the complex is detected in the form of a single spot at 0.7 cm of the deposit (migration toward the anode). When the BMP-7 is alone or does not form a complex with the polymer, it is detected at the site of the deposit and has not therefore migrated.

The results are summarized in the following table.

| Polymer | Migration |
|---|---|
| None | No |
| Polymer 1 | Yes |
| Polymer 2 | Yes |
| Polymer 3 | Yes |
| Polymer 4 | Yes |
| Polymer 5 | Yes |
| Polymer 6 | Yes |
| Polymer 7 | Yes |
| Polymer 9 | Yes |
| Polymer 12 | Yes |
| Polymer 13 | Yes |
| Polymer 14 | Yes |
| Polymer 15 | Yes |
| Polymer 17 | Yes |
| Polymer 18 | Yes |
| Polymer 19 | Yes |
| Polymer 20 | Yes |
| Polymer 22 | Yes |
| Polymer 23 | Yes |
| Polymer 24 | No |

### Example 27

### Solubilization of BMP-7 at Neutral pH at a Polymer/BMP-7 Mass Ratio of 10

A test of solubilization of Bone Morphogenetic Protein 7 (BMP-7) was developed in order to demonstrate the solubilizing power of various polymers at physiological pH. BMP-7 is soluble at acid pH and has a very low solubility limit at physiological pH, of about a few micrograms/mL.

The polymers described in this application are used in this test. The test consists in using a BMP-7 solution at acid pH, for example a 10 mM lactate buffer at pH 3. The BMP-7 is at an initial concentration of 2.47 mg/ml. 2.02 mL of this BMP-7 solution are mixed with 2.7 mL of a solution of polymer at 18.5 mg/mL containing 18 mM of phosphate buffer at pH 7.4. After mixing, the final pH is adjusted to physiological pH by adding a mixture of 1N sodium hydroxide and water so as to obtain a final formulation volume of 5 mL. The formulations are analyzed by visual observation, turbidity and dynamic light scattering in order to detect the presence of aggregates.

The results for the various solutions are collated in the following table.

| | [polymer] | [BMP-7] | | |
|---|---|---|---|---|
| Polymer | mg/ml | mg/ml | solubility | pH |
| None | | 1 | No | 7.4 |
| Polymer 2 | 10 | 1 | Yes | 7.4 |
| Polymer 3 | 10 | 1 | Yes | 7.4 |
| Polymer 4 | 10 | 1 | Yes | 7.4 |
| Polymer 5 | 10 | 1 | Yes | 7.4 |
| Polymer 7 | 10 | 1 | Yes | 7.4 |
| Polymer 8 | 10 | 1 | Yes | 7.4 |
| Polymer 9 | 10 | 1 | Yes | 7.4 |
| Polymer 24 | 10 | 1 | No | 7.4 |

This test makes it possible to demonstrate the improvement in the solubilization of BMP-7 at physiological pH by means of this new family of polymers. On the other hand, the unmodified sodium dextranmethylcarboxylate, even at a concentration of 30 mg/mL, does not enable a clear solution of BMP-7 to be obtained.

### Example 28

### Solubilization of BMP-7 at Neutral pH at a Polymer/BMP-7 Mass Ratio of 4

A test of solubilization of Bone Morphogenetic Protein 7 (BMP-7) was developed in order to demonstrate the solubilizing power of various polymers at physiological pH. BMP-7 is soluble at acid pH and has a very low solubility limit at physiological pH, of the order of a few micrograms/mL.

The polymers described in this application are used in this test. By way of comparison, two polymers described in patent application FR0702316 are also used:

Counterexample 1: sodium dextranmethylcarboxylate modified with phenylalanine,
Counterexample 2: sodium dextranmethylcarboxylate modified with leucine.

The test consists in using a BMP-7 solution at acid pH, for example a 10 mM lactate buffer at pH 3. The BMP-7 is at an initial concentration of 2.47 mg/ml. 2.02 mL of this BMP-7 solution are mixed with 2.7 mL of a solution of polymer at 7.3 mg/mL containing 18 mM of phosphate buffer at pH 7.4. After mixing, the final pH is adjusted to physiological pH by adding a mixture of 1N sodium hydroxide and water so as to obtain a final formulation volume of 5 mL. The formulations are analyzed by visual observation, turbidity and dynamic light scattering in order to detect the presence of aggregates.

The results for the various solutions are collated in the following table.

| | [Polymer] | [BMP-7] | | |
|---|---|---|---|---|
| Solution | mg/ml | mg/ml | solubility | pH |
| None | 4 | 1 | No | 7.4 |
| Polymer 2 | 4 | 1 | Yes | 7.4 |
| Polymer 5 | 4 | 1 | Yes | 7.4 |
| Polymer 17 | 4 | 1 | Yes | 7.4 |
| Polymer 19 | 4 | 1 | Yes | 7.4 |
| Polymer 21 | 4 | 1 | Yes | 7.4 |
| Polymer 24 | 4 | 1 | No | 7.4 |
| Counterexample | 4 | 1 | No | 7.4 |
| 1-FR0702316 | | | | |
| Counterexample | 4 | 1 | No | 7.4 |
| 2-FR0702316 | | | | |

This test makes it possible to demonstrate the improvement in the solubilization of BMP-7 at physiological pH by means of this new family of polymers. On the other hand, the unmodified sodium dextranmethylcarboxylate, even at a concentration of 30 mg/mL, does not enable a clear solution of BMP-7 to be obtained.

### Example 29

### Solubilization of BMP-7 at Neutral pH at a Polymer/BMP-7 Mass Ratio of 1

A test of solubilization of BMP-7 was developed in order to demonstrate the solubilizing power of various polymers at physiological pH and for polymer/BMP-7 mass ratios of 1.

The test consists in using a BMP-7 solution at acid pH, for example a 10 mM lactate buffer at pH 3. The BMP-7 is at an initial concentration of 2.47 mg/ml. 2.02 mL of this BMP-7 solution are mixed with 2.8 mL of a solution of polymer at 1.8 mg/mL containing 18 mM of phosphate buffer at pH 7.4. After mixing, the final pH is adjusted to physiological pH by adding a mixture of 1N sodium hydroxide and water so as to obtain a final formulation volume of 5 mL. The formulations are analyzed by visual observation, turbidity and dynamic light scattering in order to detect the presence of aggregates. The results show that Polymer 1 and Polymer 5 make it possible to completely solubilize BMP-7 at physiological pH for a polymer/BMP-7 mass ratio of 1.

### Example 30

### Solubilization of the BMP-2 Lyophilisate at a Polymer/BMP-2 Mass Ratio of 10

A test of solubilization of a Bone Morphogenetic Protein 2 (BMP-2) lyophilisate was developed in order to demonstrate the solubilizing power of various polymers at physiological pH. The BMP-2 is solubilized in a buffer containing sucrose (Sigma), glycine (Sigma), glutamic acid (Sigma), sodium chloride (Riedel-de-Haën) and polysorbate 80 (Fluka). This solution is adjusted to pH 4.5 by adding sodium hydroxide and is then lyophilized. 283.2 mg of lyophilisate contain approximately 12 mg of BMP-2.

The polymers described in this application are used in this test.

The test consists in introducing around exactly 14.5 mg of lyophilisate containing 0.62 mg of BMP-2 into a 1 mL flask. The lyophilisate is then taken up with 410 μL of a solution so as to achieve a final BMP-2 concentration of 1.5 mg/mL at physiological pH. The visual appearance of the solution is recorded after stirring for 15 minutes at a low speed on a roll.

The results for various solutions with BMP-2 are collated in the following table.

| | [polymer] | [BMP-2] | | |
|---|---|---|---|---|
| Solution | mg/ml | mg/ml | solubility | pH |
| Phosphate | 0 | 1.5 | No | 7.4 |
| buffer | | | | |
| Polymer 5 | 15 | 1.5 | Yes | 7.4 |
| Polymer 9 | 15 | 1.5 | Yes | 7.4 |
| Polymer 10 | 15 | 1.5 | Yes | 7.4 |
| Polymer 11 | 15 | 1.5 | Yes | 7.4 |
| Polymer 19 | 15 | 1.5 | Yes | 7.4 |
| Polymer 24 | 15 | 1.5 | No | 7.4 |
| Water | 15 | 1.5 | Yes | 4.5 |

### Example 31

### Solubilization of a BMP-2 Lyophilisate at a Polymer/BMP-2 Mass Ratio of 6.25

A test of solubilization of a Bone Morphogenetic Protein 2 (BMP-2) lyophilisate was developed in order to demonstrate the solubilizing power of various polymers at physiological pH. The BMP-2 is solubilized in a buffer containing sucrose (Sigma), glycine (Sigma), glutamic acid (Sigma), sodium chloride (Riedel-de-Haën) and polysorbate 80 (Fluka). The pH of this solution is adjusted to pH 4.5 by adding sodium hydroxide and then the solution is lyophilized. 283.2 mg of lyophilisate contain approximately 12 mg of BMP-2.

The polymers according to the invention are used in this test. By way of comparison, a polymer described in patent application FR0702316 is also used in this test, sodium dextranmethylcarboxylate modified with ethyl phenylalaninate.

The test consists in introducing around exactly 4 mg of lyophilisate containing 0.168 mg of BMP-2. The lyophilisate is then taken up with 210 μL of an aqueous solution so as to achieve a final BMP-2 concentration of 0.8 mg/mL at physiological pH, the final polymer concentration being 5 mg/ml.

The visual appearance of the solution is recorded after stirring for 5 minutes at a low speed on a roll.

The results for various solutions are collated in the following table.

| | [Polymer] | [BMP-2] | | |
|---|---|---|---|---|
| Solution | mg/ml | mg/ml | Solubility | pH |
| Water | 5 | 0.8 | Yes | 4.3 |
| Polymer 1 | 5 | 0.8 | Yes | 7.4 |
| Polymer 5 | 5 | 0.8 | Yes | 7.5 |
| Polymer 25 | 5 | 0.8 | Yes | 7.4 |
| Counterexample | 5 | 0.8 | No | 7.5 |
| FR0702316 | | | | |

The addition of water results in a clear BMP-2 solution but at an acid pH.

This test makes it possible to demonstrate the improvement in the solubilization of BMP-2 at physiological pH by means of the polymers according to the invention. On the other hand, sodium dextranmethoxycarbamate modified with ethyl phenylalaninate does not enable a clear BMP-2 solution to be obtained.

### Example 32

### Lyoprotectant Effect of the Polymers on BMP-7

In order to test the ability of the polymers to maintain the integrity of BMP-7, a test of lyophilization of these formulations was carried out. Lyophilization is a process which stresses proteins and which often results in aggregation of the protein during the process. By way of example, the formulation obtained in example 27 with polymer 5 was lyophilized. This lyophilisate was then reconstituted with injectable water at the initial concentration. The solution was then analyzed and compared with the initial solution by dynamic light scattering. The analysis shows that the two solutions are identical and therefore that the lyophilization has not introduced any aggregation of the protein.

### Example 33

### Solubilization of BMP-7 at Physiological pH and at a Concentration above 1 mg/ml

The objective of this test is to solubilize BMP-7, at physiological pH, at a concentration above 1 mg/ml.

A volume of 5.5 mL of a solution of BMP-7 at 2 mg/ml and acid pH is mixed with 5.5 mL of a solution of polymer 2 at a concentration of 6.9 mg/ml so as to obtain a solution of BMP-7 at 1 mg/mL containing 3.45 mg/mL of polymer 1. This solution is then lyophilized by means of a conventional lyophilization process.

The solution is then reconstituted with a 10 mM phosphate buffer solution and adjusted to physiological pH by adding a 1N sodium hydroxide solution so as to obtain a formulation in which the BMP-7 concentration is 5 mg/mL and the polymer concentration is 17.3 mg/mL.

The resulting solution is completely clear, which does not suggest the presence of aggregates, thereby confirming the dynamic light scattering analysis.

### Example 34

### Stability of the BMP-7 Formulation with Dilution

The aim of this test is to simulate the injection of a formulation into a biological medium, for instance in the case of a subcutaneous or intravenous administration to humans or to an animal. Specifically, after injection, the formulation undergoes a dilution with a biological fluid having a pH of 7.4.

A formulation of BMP-7 at acid pH (pH 3) at 1 mg/ml is injected into a PBS buffer at pH 7.4 with a dilution factor of 10. During the injection, turbidity of the solution is observed, resulting from the precipitation of the protein. This aggregation of BMP-7 in the PBS is confirmed by a dynamic light scattering measurement.

In this same test, if a formulation as described in example 14 with one of the polymers 1 to 11 at a comparable BMP-7 concentration (i.e. 1 mg/ml) is used, no turbidity is observed. The dynamic light scattering measurement demonstrates an absence of aggregates in this sample.

The BMP-7/polymer formulation therefore has the advantage of being soluble and liquid at physiological pH, but also of being capable of withstanding dilution at physiological pH while preventing aggregation phenomena, which may be particularly advantageous in the context of the development of a pharmaceutical product for injection.

### Example 35

### Immunogenicity of Two BMP-7 Compositions

It has been demonstrated that several animal species can be predictive of the immunological activity of BMP-7. Among these preclinical models are the rabbit posterolateral fusion model. This model was retained for evaluating the reduction in the immunological effect of BMP-7 in the form of a complex with the polymers of the invention (OP-1 Immunogenicity Report, FDA StrykerBiotech Briefing for Mar. 31, 2009 Advisory Committee Meeting).

The posterolateral lumbar fusion model (arthrodesis at L5-L6) was performed on rabbits according to the experimental protocol described in patent WO 2010/058106. The rabbits were divided up into two groups, each of 4 rabbits, the first group was implanted with two collagen sponges containing BMP-7 alone (650 μg), Implant 1, the second group was implanted with two collagen sponges containing a BMP-7 complex with a polymer (650 μg of BMP-7), Implant 2.

Implant 1 was prepared by depositing 800 μL of a solution of BMP-7 in a 5% lactose buffer, pH 3.5, at a concentration of 0.81 mg/mL, i.e. a BMP-7 dose of 650 μg in a crosslinked collagen type I sponge having a volume of 2250 μL.

Implant 2 was obtained after successive impregnations of a crosslinked collagen type I sponge having a volume of 2250 μL with 400 μL of a solution containing BMP-7 at 1.63 mg/mL, i.e. 650 μg of BMP-7, polymer 1 at 20 mg/mL, i.e. 8 mg of polymer 1, sodium phosphate at 0.23 M, i.e. 92 μmol, and sodium bicarbonate at 0.62 M, i.e. 248 μmol, and then with 400 μL of a solution containing calcium chloride at 0.38 M, i.e. 153 μmol. Each solution is left in contact with the sponge for 15 minutes after addition. After these impregnation periods, the sponge is ready for the implantation.

Serum collections were performed before implantation (day 0) and at days 10, 32, 39 and 68 after surgery on all the animals. The samples were stored at −80° C. The concentration of rabbit IgG antibodies directed against rhBMP-7 in the rabbit sera collected was measured by means of an ELISA assay according to the protocol described in the article (A. R. Mire-Sluis et al., J. Immunol. Methods 2004, 289 (1-2), 1-16). rhBMP-7 at 2 μg/mL in phosphate buffer saline (PBS) is adsorbed onto the assay plate at 4° C. overnight. The plate is washed twice with PBS, saturated with a solution of PBS containing 1% of bovine serum albumin (BSA), and washed 3 times with PBS containing 0.06% of tween 20. The rabbit sera are diluted to 1/40 in PBS containing 0.1% of BSA and 0.06% of tween 20. The positive control is a solution of a rabbit anti-rhBMP-7 antibody of IgG isotype (supplier Peprotech, reference 500-P198). The negative control is a mixture of 20 sera from healthy untreated rabbits. The detection antibody is a donkey anti-rabbit IgG antiserum coupled to alkaline phosphatase (supplier Cliniscience, reference 6440-05). The antibody detection threshold of this test is 160 ng/mL with 5% of false positives. The intraplate and interplate variabilities are less than 5%.

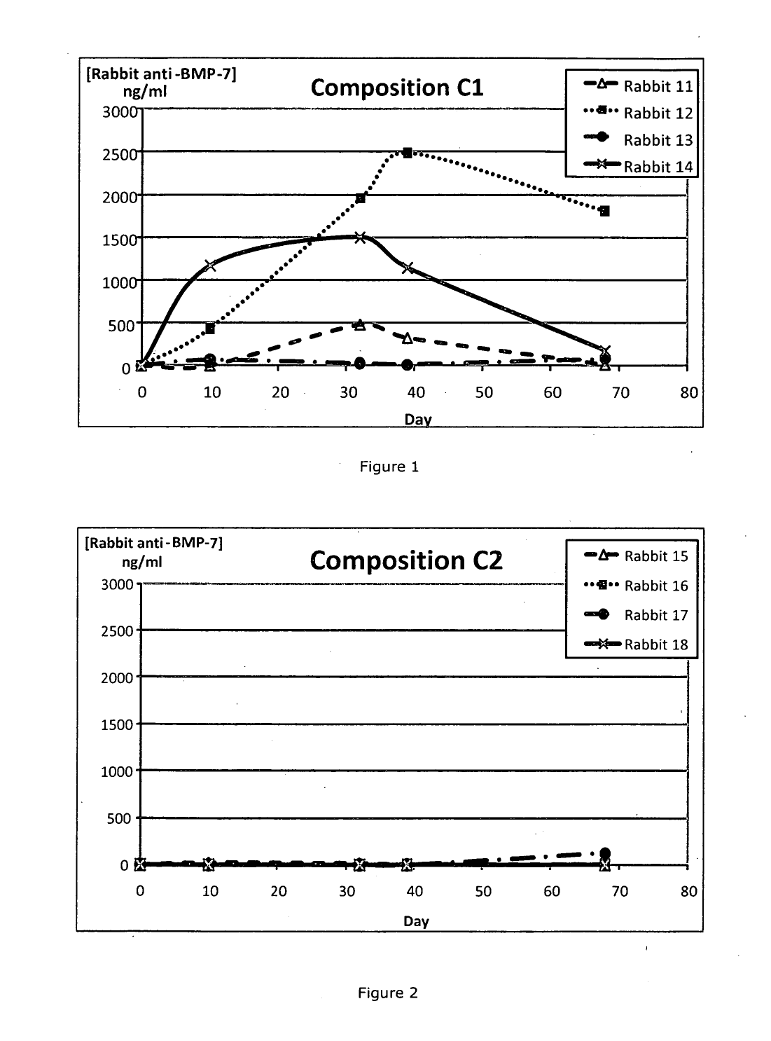

The results show a large increase in the anti-rhBMP-7 IgG titer measured for two rabbits (rabbits 12 and 14), a moderate transient increase for one rabbit (rabbit 11) and an absence of response for one rabbit (rabbit 13) for composition C1. The anti-rhBMP-7 IgG titer observed with composition C2 is below the detection threshold at all the times, see table below and FIGS. 1 and 2.

TABLE 1
Concentration of anti-rhBMP-7 IgG measured in the sera of
rabbits at several times after implantation of 2 collagen
sponges soaked with composition C1 or with composition C2.

| C1 | rabbit 11 | rabbit 12 | rabbit 13 | rabbit 14 |
|---|---|---|---|---|
| day 0 | <dt | <dt | <dt | <dt |
| day 10 | <dt | 434.12 | <dt | 1167.27 |
| day 32 | 480.18 | 1951.95 | <dt | 1499.10 |
| day 39 | 325.58 | 2483.66 | <dt | 1139.16 |
| day 68 | <dt | 1807.51 | 178.02 | <dt |
| C2 | rabbit 15 | rabbit 16 | rabbit 17 | rabbit 18 |
| day 0 | <dt | <dt | <dt | <dt |
| day 10 | <dt | <dt | <dt | <dt |
| day 32 | <dt | <dt | <dt | <dt |
| day 39 | <dt | <dt | <dt | <dt |
| day 68 | <dt | <dt | <dt | <dt |

1. A polysaccharide/BMP complex, the BMP being chosen from the group consisting of BMP-2 and BMP-7, soluble at physiological pH, wherein the polysaccharide/BMP mass ratio is less than 15, the polysaccharide being chosen from the group of polysaccharides comprising carboxyl functional groups, at least one of which is substituted with at least one hydrophobic radical, denoted Ah:
said hydrophobic radical Ah being a residue of a hydrophobic compound chosen from hydrophobic alcohols or acids comprising a linear, branched or cyclic alkyl chain containing at least 6 carbon atoms, said hydrophobic radical Ah being bonded to a linker arm R by a function G resulting from coupling between at least one reactive function of said hydrophobic compound and a reactive function of the linker arm precursor R′,
said linker arm R being bonded to the polysaccharide by a bond F resulting from coupling between a reactive function of the linker arm precursor R′ and a carboxyl function of the anionic polysaccharide, R being an at least divalent radical consisting of a chain comprising between 1 and 15 carbons, which is optionally branched and/or unsaturated, optionally comprising one or more heteroatoms, such as O, N and/or S, and resulting from a precursor R′ having at least two reactive functions, at least one being an amine function and the others, which are identical or different, being chosen from the group consisting of alcohol, acid or amine functions,
F being an amide function,
G being either an amide, ester or carbamate function,
the unsubstituted carboxyl functions of the anionic polysaccharide being in the form of a cation carboxylate, preferably an alkali metal cation such as Na⁺ or K⁺, said polysaccharide comprising carboxyl functional groups which are amphiphilic at neutral pH,
the BMP being chosen from the group consisting of recombinant human BMP-2 and BMP-7, and homologs thereof.

2. The polysaccharide/BMP complex as claimed in claim 1, wherein the BMP is chosen from the group consisting of recombinant human BMP-2s and homologs thereof.

3. The polysaccharide/BMP complex as claimed in claim 1, wherein the BMP is chosen from the group consisting of recombinant human BMP-7s and homologs thereof.

4. The complex as claimed in claim 1, wherein the polysaccharides comprising carboxyl functional groups are synthetic polysaccharides obtained from neutral polysaccharides, onto which at least 15 carboxyl functional groups per 100 saccharide units have been grafted, of general formula I: 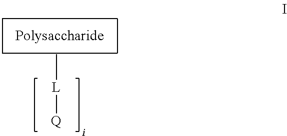
the natural polysaccharides being chosen from the group of polysaccharides of which the bonds between the glycosidic monomers comprise (1,6)-bonds,
L being a bond resulting from coupling between a precursor of the linker arm Q and an —OH function of the polysaccharide and being either an ester, carbamate or ether function,
i represents the molar fraction of the substituents L-Q per saccharide unit of the polysaccharide,
Q being chosen from the radicals of general formula II: 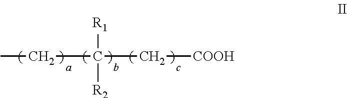
in which:
1≦a+b+c≦6, and
0≦a≦3,
0≦b≦3
0≦c≦3,
R₁ and R₂, which may be identical or different, are chosen from the group consisting of —H, linear or branched C₁ to C₃ alkyl, —COOH and the radical 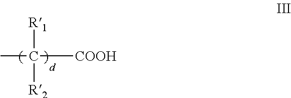
of formula III in which
1≦d≦3, and
R′₁ and R′₂, which may be identical or different, are chosen from the group consisting of —H and a linear or branched C₁ to C₃ alkyl group.

5. The complex as claimed in claim 1, wherein the polysaccharide is chosen from the group consisting of polysaccharides of which the bonds between the glycosidic monomers comprise (1,6)-bonds.

6. The complex as claimed in claim 5, wherein the polysaccharide is chosen from the group consisting of dextran and pullulan.

7. The complex as claimed in claim 1, wherein the polysaccharide is chosen from the polysaccharides of formula IV: 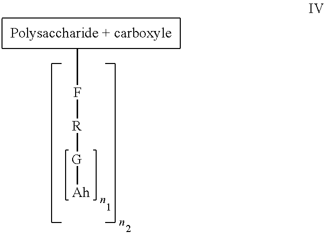
Ah being a residue of a hydrophobic compound chosen from hydrophobic alcohols or acids comprising a linear, branched or cyclic alkyl chain containing at least 6 carbon atoms, produced from coupling between a hydroxide or acid function of the hydrophobic compound and a reactive function of the precursor R′ of R,
F being an amide function,
G being either an ester function, or a carbamate function, or an amide function,
R being an at least divalent radical consisting of a chain comprising between 1 and 15 carbons, which is optionally branched and/or unsaturated, optionally comprising one or more heteroatoms, such as O, N and/or S, resulting from a precursor R′ having at least two reactive functions, at least one being an amine function and the others, which are identical or different, being chosen from the group consisting of alcohol, acid or amine functions,
n₁ being equal to 1 or 2,
n₂ representing the molar fraction of the carboxyl functions of the polysaccharide which are substituted with F—R-G-Ah and being between 0.01 and 0.7, and
when the carboxyl function of the polysaccharide is not substituted with F—R-G-Ah, then the carboxyl functional group(s) of the polysaccharide are cation carboxylates, preferably an alkali cation such as Na⁺ or K⁺.

8. The complex as claimed in claim 1, wherein Ah is a hydrophobic alcohol.

9. The complex as claimed in claim 8, wherein the hydrophobic alcohol is chosen from alcohols consisting of a branched or unbranched, saturated or unsaturated alkyl chain comprising from 6 to 18 carbons.

10. The complex as claimed in claim 9, wherein the hydrophobic alcohol is octanol.

11. The complex as claimed in claim 9, wherein the hydrophobic alcohol is dodecanol.

12. The complex as claimed in claim 9, wherein the hydrophobic alcohol is 2-ethylbutanol or isohexanol.

13. The complex as claimed in claim 1, wherein Ah is a hydrophobic acid.

14. The complex as claimed in claim 13, wherein the hydrophobic acid is chosen from fatty acids.

15. The complex as claimed in claim 14, wherein the fatty acids are chosen from the group consisting of acids consisting of a branched or unbranched, saturated or unsaturated alkyl chain comprising from 6 to 50 carbons.

16. The complex as claimed in claim 1, wherein the polymer/BMP mass ratio is less than or equal to 10.

17. The complex as claimed in claim 1, wherein the polymer/BMP mass ratio is less than or equal to 5.

18. The complex as claimed in claim 1, wherein is chosen from the group consisting of the following complexes:
40 kDa sodium dextranmethylcarboxylate modified with octanol phenylalaninate/BMP-2, mass ratio=10.
40 kDa sodium dextranmethylcarboxylate modified with dihexanol aspartate/BMP-2, mass ratio=10.
10 kDa dextranmethylcarboxylate modified with N-[2-((2-octanoylamino-3-phenyl)propanoylamino)]ethanamine/BMP-2, mass ratio=10.
40 kDa dextranmethylcarboxylate modified with N-(2-aminoethyl)dodecanamide/BMP-2, mass ratio=10.
40 kDa dextranmethylcarboxylate modified with octanol leucinate, mass ratio=10.
40 kDa sodium dextranmethylcarboxylate modified with dodecanol glycinate, mass ratio=10.
10 kDa sodium dextranmethylcarboxylate modified with octanol glycinate/BMP-2, mass ratio=6.25.
10 kDa dextranmethylcarboxylate modified with octanol phenylalaninate/BMP-2, mass ratio=6.25.
40 kDa dextranmethylcarboxylate modified with dodecanol alaninate/BMP-2, mass ratio=6.25.

19. The complex as claimed in claim 1, wherein is chosen from the group consisting of the following complexes:
40 kDa sodium dextranmethylcarboxylate modified with octanol glycinate/BMP-7, mass ratio=10.
40 kDa sodium dextranmethylcarboxylate modified with octanol glycinate/BMP-7, mass ratio=12.3.
10 kDa sodium dextranmethylcarboxylate modified with octanol glycinate/BMP-7, mass ratio=10.
10 kDa sodium dextranmethylcarboxylate modified with octanol glycinate/BMP-7, mass ratio=4.
10 kDa sodium dextranmethylcarboxylate modified with dodecanol glycinate/BMP-7, mass ratio=10.
10 kDa sodium dextranmethylcarboxylate modified with isohexanol leucinate/BMP-7, mass ratio=10.
40 kDa sodium dextranmethylcarboxylate modified with octanol phenylalaninate/BMP-7, mass ratio=10.
40 kDa sodium dextranmethylcarboxylate modified with octanol phenylalaninate/BMP-7, mass ratio=4.
40 kDa sodium dextranmethylcarboxylate modified with octanol valinate/BMP-7, mass ratio=10.
40 kDa sodium dextranmethylcarboxylate modified with ethanolamine laurate ester/BMP-7, mass ratio=10.
40 kDa sodium dextranmethylcarboxylate modified with dihexanol aspartate/BMP-7, mass ratio=10.
10 kDa dextranmethylcarboxylate modified with cholesterol leucinate/BMP-7, mass ratio=10.
10 kDa dextranmethylcarboxylate modified with octanol phenylalaninate/BMP-7, mass ratio=10.
10 kDa dextranmethylcarboxylate modified with 3,7-dimethyl-1-octanol phenylalaninate/BMP-7, mass ratio=10.
10 kDa dextranmethylcarboxylate modified with 2-(2-aminoethoxy)ethyl octanoate/BMP-7, mass ratio=10.
10 kDa dextranmethylcarboxylate modified with 2-(2-aminoethoxy)ethyl dodecanoate/BMP-7, mass ratio=10.
10 kDa dextranmethylcarboxylate modified with N-[2-((2-octanoylamino-3-phenyl)propanoylamino)]ethanamine/BMP-7, mass ratio=10.
10 kDa dextranmethylcarboxylate modified with N-[2-((2-octanoylamino-3-phenyl)propanoylamino)]ethanamine/BMP-7, mass ratio=4.
10 kDa dextranmethylcarboxylate modified with N-(2-aminoethyl)octanamide/BMP-7, mass ratio=10.
40 kDa dextranmethylcarboxylate modified with N-(2-aminoethyl)dodecanamide/BMP-7, mass ratio=10.
40 kDa dextranmethylcarboxylate modified with N-(2-aminoethyl)dodecanamide/BMP-7, mass ratio=4.
10 kDa sodium dextranmethylcarboxylate modified with didodecanol aspartate/BMP-7, mass ratio=10.
10 kDa dextran carbamate N-methyl(sodium carboxylate) modified with N-(2-aminoethyl)dodecanamide/BMP-7, mass ratio=4.
10 kDa dextranmethylcarboxylate modified with isohexanol phenylalaninate/BMP-7, mass ratio=10.
10 kDa dextranmethylcarboxylate modified with benzyl phenylalaninate/BMP-7, mass ratio=10.
10 kDa dextranmethylcarboxylate modified with isohexanol phenylalaninate/BMP-7, mass ratio=10.

20. A therapeutic composition, which comprises an amphiphilic polysaccharide/BMP-7 complex as claimed in claim 1.

21. A therapeutic composition, which comprises an amphiphilic polysaccharide/BMP-2 complex as claimed in claim 1.
